(19) United States
(12) Patent Application Publication
(10) Pub. No.: US 2015/0066113 A1
(43) Pub. Date: Mar. 5, 2015

(54) Radiolucent Metal Case Plate to Facilitate Communications in an Implantable Medical Device

(71) Applicant: Boston Scientific Neuromodulation Corporation, Valencia, CA (US)

(72) Inventor: Jeffrey Van Funderburk, Woodland Hills, CA (US)

(21) Appl. No.: 14/469,739

(22) Filed: Aug. 27, 2014

Related U.S. Application Data
(60) Provisional application No. 61/874,186, filed on Sep. 5, 2013.

(51) Int. Cl. A61N 1/372; A61N 1/378

(52) U.S. Cl. CPC A61N 1/37223; A61N 1/3787
USPC 607/60

(57) ABSTRACT

Disclosed is an improved case for an implantable medical device having an internal communication coil in which a lower-conductivity, more-radiolucent metallic plate is provided proximate to the coil. The remainder of the case can be formed of a higher-conductivity metallic material which is easier to form and thus lends itself to the manufacture of implantable medical devices with smaller cases for example. As both the plate and the remainder of the case are metallic, they can be easily joined by reliable laser welding techniques for example.

### CROSS-REFERENCE TO RELATED APPLICATIONS

This is a non-provisional of U.S. Provisional Ser. No. 61/874,186, filed Sep. 5, 2013, which is incorporated herein by reference in its entirety, and to which priority is.

### FIELD OF THE INVENTION

The present invention relates to improving wireless communications in an implantable medical device such as an implantable pulse generator.

### BACKGROUND

Implantable stimulation devices deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any implantable medical device or in any implantable medical device system.

As shown in FIG. 1, a SCS system includes an Implantable Pulse Generator (IPG) 10, whose construction is further described in a Provisional Patent Application entitled “Construction for an Implantable Medical Device Employing an Internal Support Structure,” Ser. No. 61/874,194, which is concurrently filed herewith, and which is incorporated herein by reference in its entirety. IPG 10 includes a biocompatible device case 30 that holds the circuitry and battery 34 (FIG. 2) necessary for the IPG to function. The IPG 10 is coupled to electrodes 16 via one or more electrode leads (two such leads 14a and 14b are shown), such that the electrodes 16 form an electrode array 12. The electrodes 16 are carried on a flexible body 18, which also houses the individual signal wires 20 coupled to each electrode. The signal wires 20 are connected to the IPG 10 at one or more lead connectors 24a and 24b fixed in a header 28, which can comprise epoxy for example. In the illustrated embodiment, there are eight electrodes on lead 14a, labeled E1-E8, and eight electrodes on lead 14b, labeled E9-E16, although the number of leads and electrodes is application specific and therefore can vary. In a SCS application, electrode leads 14a and 14b are typically implanted on the right and left side of the dura within the patient's spinal cord. The proximal ends 22a and 22b of leads 14a and 14b are then tunneled through the patient's flesh to a distant location, such as the buttocks, where the IPG case 30 is implanted, at which point they are coupled to the lead connector(s) 24a and 24b.

FIG. 2 shows IPG 10 with case 30 removed so that internal components can be seen. Of particular importance is the communication coil (antenna) 40, which enables communication between the IPG 10 and a device external to the patient, such as a hand-holdable portable external controller 100. External controller 100 itself contains a communication coil (antenna; not shown), thus allowing bidirectional communication 102 to occur by magnetic induction between the two devices. This is useful as it allows a user to interface with the external controller 100 to adjust the therapy that the IPG 10 is providing (e.g., to increase or decrease the stimulation being provided, to change which electrodes are providing the stimulation, etc.), and also to review status information reported by the IPG. Further details concerning an external controller and how it communicates with an IPG can be found in U.S. Provisional Patent Application Ser. No. 61/773,476, filed Mar. 6, 2013, which is incorporated herein by reference in its entirety, and with which the reader is assumed familiar.

FIG. 3 shows IPG 10 in cross section, and shows further details of construction. The majority of the room inside the case 30 is taken up by the battery 34, which in this example is a permanent, non-wirelessly-rechargeable battery. (Battery 34 could also be rechargeable, in which case either coil 40 or another recharging coil would be used to wirelessly receive a charging field that is rectified to charge the battery 34). The remainder of the room in the case is taken up by circuitry, including a printed circuit board (PCB 42) that contains various electronic components and receives leads from the battery 34, the communication coil 40, and the feedthrough pins 23 that ultimately couple to the electrodes 16 via the lead connectors 24. Communication coil 40 is coupled to modulation and/or demodulation circuitry 27 on the PCB to prepare data to be transmitted and decode data that is received. A support structure 38, made of plastic for example, is used to hold the communication coil 40 and the PCB 42 and to provide a mechanically-stable arrangement of the components inside the case 30, as further discussed in the above-incorporated application. Case 30 is formed in two portions 30a and 30b, each having substantially parallel top 31a and bottom 31b sides (with 5%). The case portions surround the electronics, and are laser welded together as well as laser welded to a feedthrough 32. Thereafter, the epoxy header 26 is affixed to the case 30 and formed around the lead connectors 24.

The inventor has noticed a concern with traditional IPG design, and in particular with communications to and from an IPG. As is known, wireless communications to and from communication coil 40 can be attenuated by the conductive material of the case 30. When magnetic induction is used as the means for establishing communication link 102 for example, the generated AC magnetic field will create eddy currents in the case 30, essentially acting as an unwanted sink for the energy in the field, and thus reducing the distance at which communication between the IPG 10 and external controller 100 can reliably occur. See, e.g., U.S. Pat. No. 8,457,756. Such unwanted coupling to the case is increased when the conductivity of the material used for the case 30 is increased.

Some previous IPGs 10 have thus used a lower-conductivity material for the case 30, such as Titanium alloys (Ti) Grade 23 (which contains within manufacturing tolerances 6% Aluminum and 4% Vanadium, and no more than 0.13% Oxygen), or Grade 5 (which contains within manufacturing tolerances 6% Aluminum and 4% Vanadium, and no more than 0.20% Oxygen). However, while these materials have preferred lower conductivity, they are also more brittle. This makes it more difficult to form such materials into a shape without cracking This is a problem for IPG designers, especially as IPGs reduce in size. As sizes reduce, the degree to which the case material must be bent also increases (i.e., the radii of curvature of the bends decrease), particularly at the edges 50 and corners 52 of the case 30, as best seen in the isomeric view of FIG. 1.

Some previous IPGs 10 have used non-conductive ceramic materials for the case, see, e.g., U.S. Pat. No. 7,351,921, which like lower conductivity alloys will tend to reduce attenuation of communications in IPGs using internal communication coils. However, ceramic materials are also brittle and difficult to work with. Ceramic case components further require brazing to mechanically couple them together or to other metallic components, which can be difficult to perform.

### BRIEF DESCRIPTION OF THE DRAWINGS

FIG. 1 shows an Implantable Pulse Generator (IPG) and the manner in which electrodes are affixed.

FIG. 2 shows the IPG in wireless communication with an external controller.

FIG. 3 shows the internal construction of the IPG in cross section.

FIG. 4 shows an improved IPG having a case with a metallic plate proximate the communication coil within the case.

FIGS. 5A-5C show examples of sizing of the plate relative to the sizing of the communication coil.

FIG. 6 shows a cross section of the improved IPG with the plate to facilitate communications with an external controller.

FIG. 7 shows various manners in which the plate can be affixed to the case of the IPG.

FIG. 8 shows a modified IPG having two plates on either side.

FIG. 9 shows a cross section of an improved IPG having a charging coil and a plate to facilitate charging from an external charger.

FIG. 10 shows an alternative case configuration with which the disclosed plate(s) can be used.

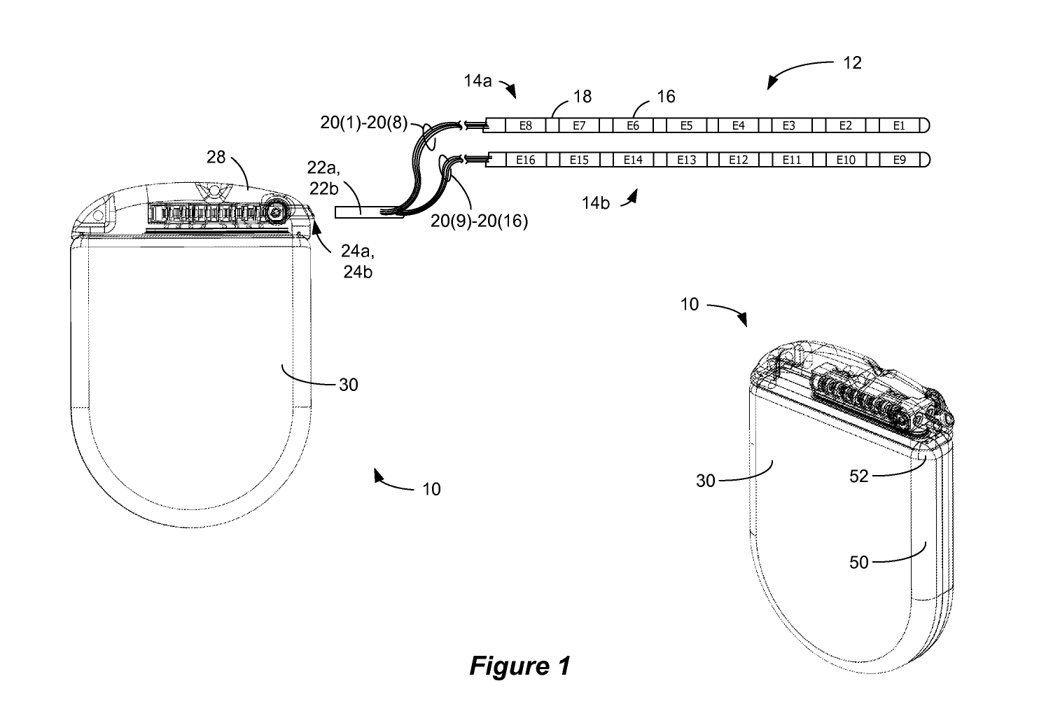

### DETAILED DESCRIPTION

The inventor discloses an improvement for a case for an implantable medical device having an internal communication coil in which a lower-conductivity, more-radiolucent metallic plate is provided proximate to the coil. The remainder of the case can be formed of a higher-conductivity metallic material which is easier to form and thus lends itself to the manufacture of implantable medical devices with smaller cases for example. As both the plate and the remainder of the case are metallic, they can be easily joined by reliable laser welding techniques for example.

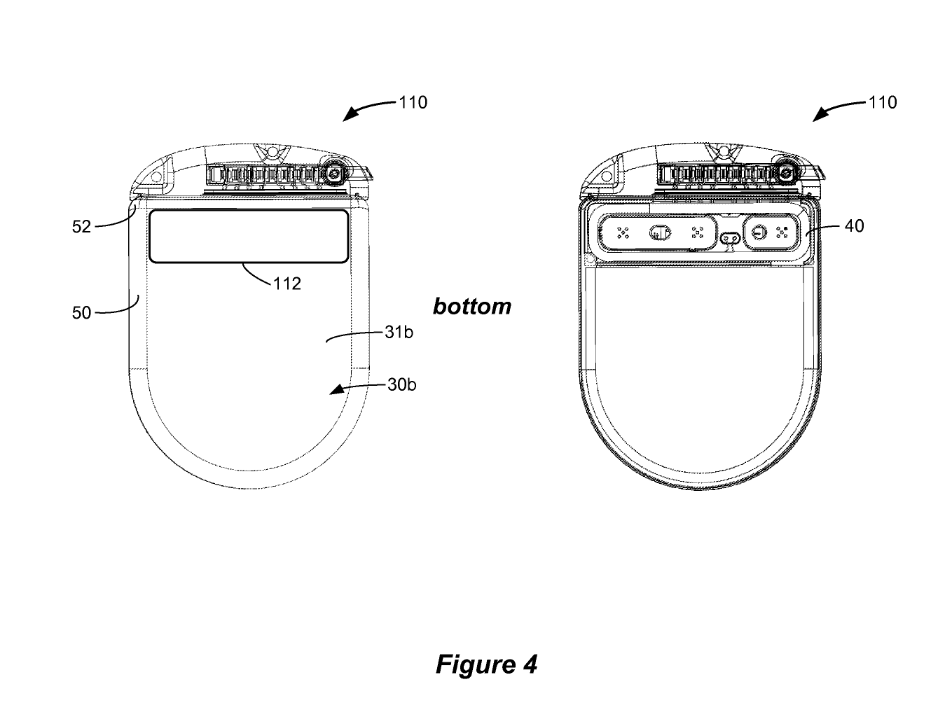

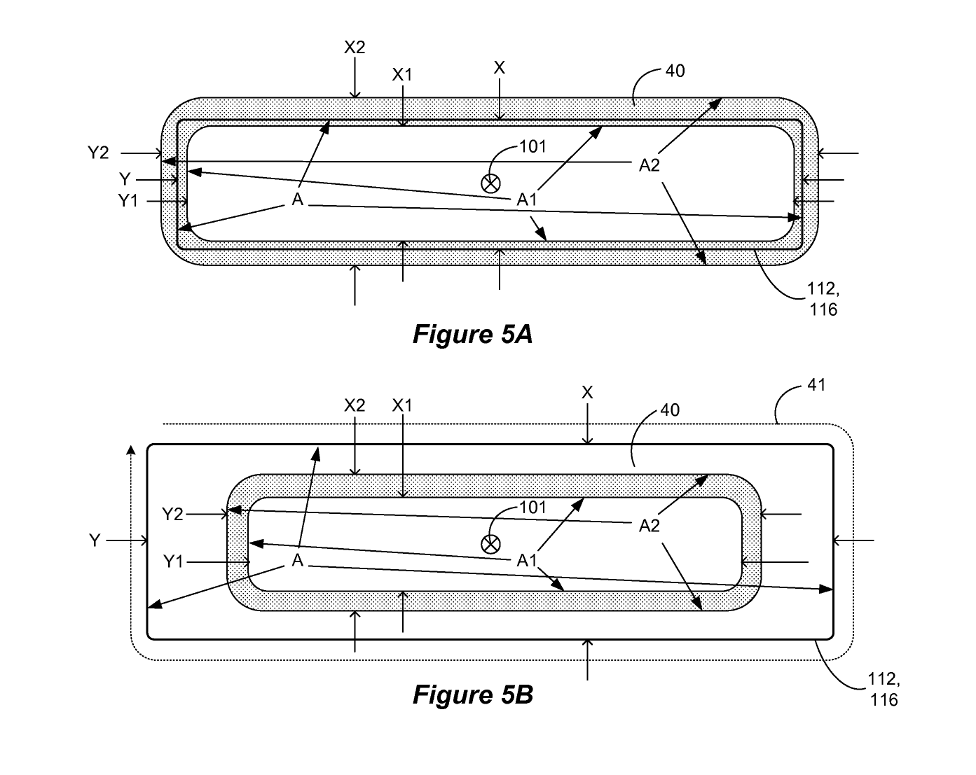

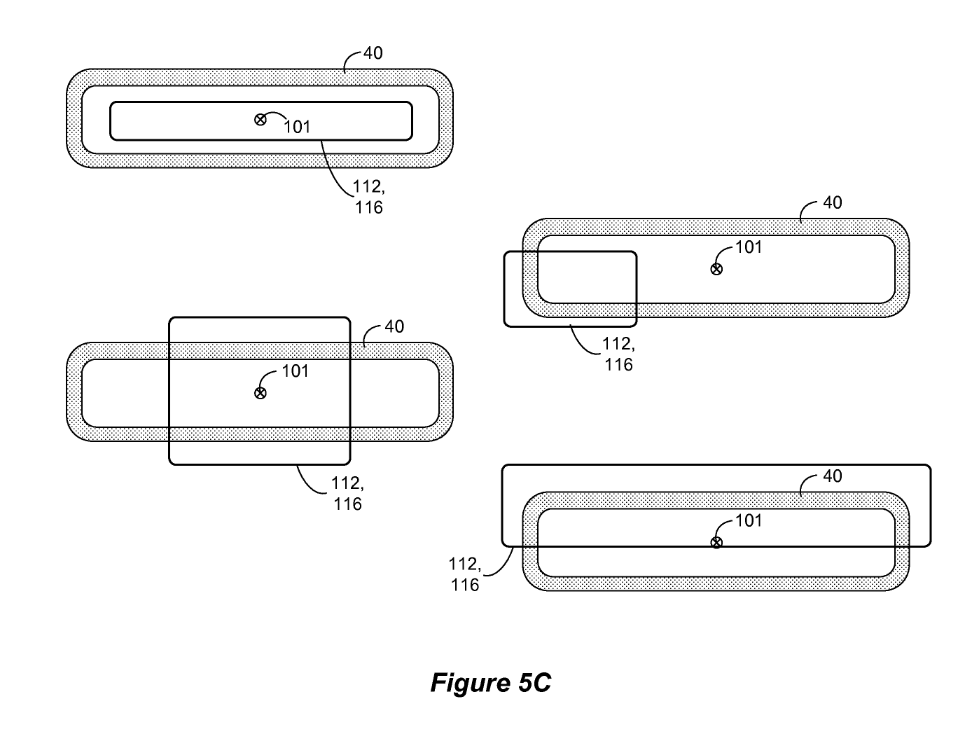

FIG. 4 shows the bottom side 31b of the improved IPG 110 (the side proximate the communication coil 40) with the bottom case portion 30b present (left) and removed (right). As shown, the bottom case portion 30b includes a metallic plate 112, a radiolucent “window” that covers a hole 116 (FIG. 5A) in the case 30 and essentially aligns with the location of the communication coil 40 within the IPG, as described further below. In this example, the plate 112 is flat, and thus does not encroach upon the curved surfaces of the bottom case portion 30b, such as the edges 50 and corners 52. This is desirable, as it is easier to affix the plate 112 to the bottom case portion 30b, as described in detail later. That being said, it is not strictly necessary that the plate 112 be flat, and it can meet with curved case portions in other examples.

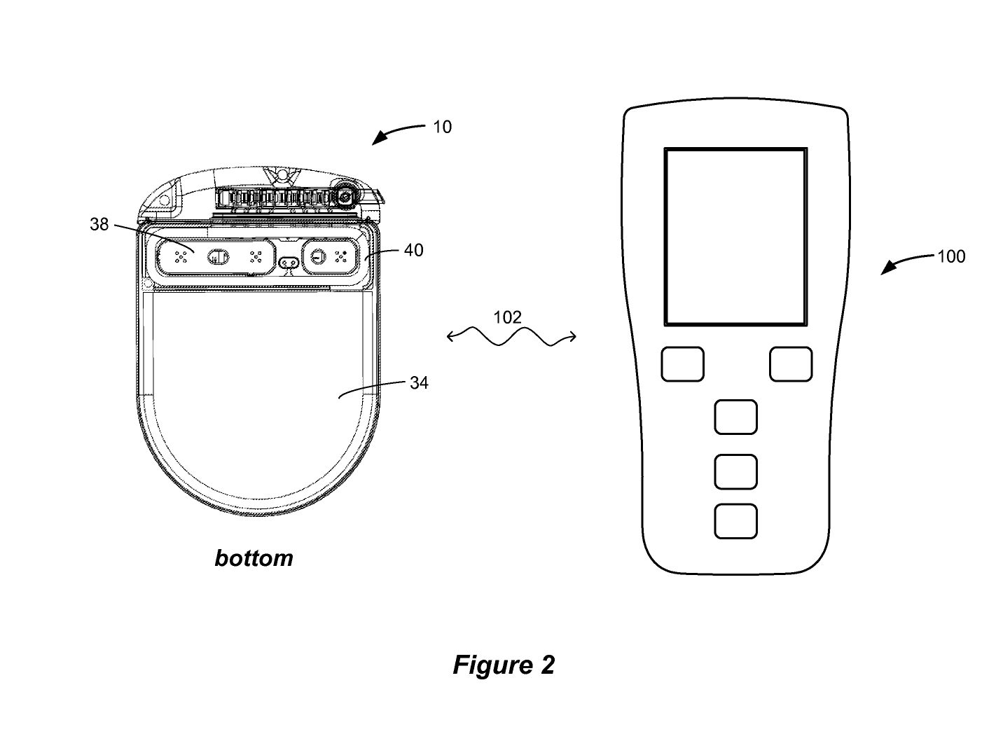

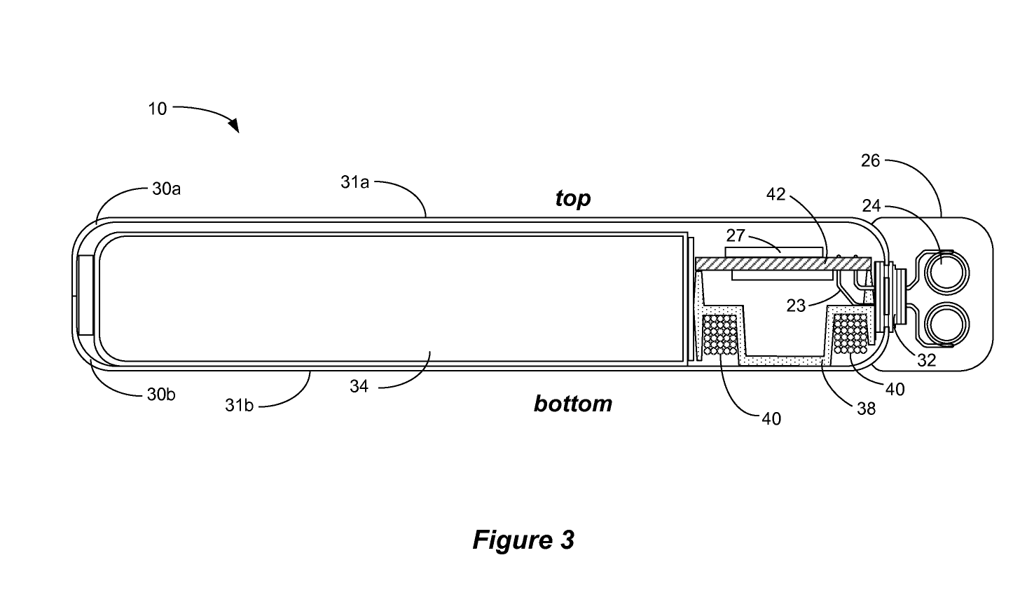

The plate 112 and bottom case portion 30b are made of two different metallic materials having different conductivities. In one non-limiting example, the bottom case portion 30b (and top case portion 30a) are formed of unalloyed TI Grades 1 (low oxygen) or 2 (standard oxygen), which consists essentially of Titanium (ignoring oxygen and other normal manufacturing impurities). This material has a higher conductivity and is less brittle than Ti Grade 23, and therefore easier to work into the three-dimensional shapes of the case portions 30a and 30b. The plate 112, by contrast, is made of a lower conductivity material such as TI Grade 23 or Grade 5. Plate 112 could also be made of other lower conductivity metallic materials as well, including other biocompatible materials not containing Titanium. Such lower-conductivity materials, as discussed earlier, are more radiolucent. When these different materials for the case portions 30a and 30b and plate 112 are used in combination, a case 30 results that is easy to form into shape, but which is less prone to attenuate wireless communications 102 to and from an external device such as external controller 100 (FIG. 2).

FIG. 5A describes further details of the size of hole 116 and/or plate 112 in the bottom case portion 30b relative to the communication coil 40 for the example IPG 110 depicted, with the size of the hole 116 and the plate 112 being discussed together because they are essentially equal in this example. As shown the communication coil 40's windings comprise outer dimensions X2 and Y2 generally defining an outer area A2, and inner dimensions X1 and Y1 generally defining an inner area A1. The hole 116/plate 112 likewise comprises dimensions X and Y generally defining an outer area A. It is preferable that the hole 116/plate 112's area A be at least equal to or larger than and overlaps the coil 40's inner area A1 (e.g., X>X1 and Y>Y1), and further are centered around an axis 101. Particularly if magnetic induction is used as the communication means for link 102, it is desired that the magnetic flux created or received inside the coil 40 be attenuated as little as possible, which the lower-conductivity plate 112 promotes, and so having A be larger than and overlapping A1 assists in this regard.

FIG. 5B shows another example for a different IPG in which the communication coil 40 has a smaller area inside the case 30, and as a result the area A of the hole 116/plate 112 can be made much larger. In particular, note that in this example the hole 116/plate 112's area A is equal to or larger than and overlaps the coil 40's outer area A2 (e.g., X>X2 and Y>Y2), and further are centered around axis 101. This provides the largest benefit, as some of the magnetic flux implicated in inductive communications appears outside of the outer area A2 of the communication coil 40, as one skilled in the art would understand.

Having as large of a hole 116/plate 112 as possible forces eddy currents 41 through the more-conductive bottom case portion 30b to take a longer more-resistive paths. As this path becomes longer, the less-conductive plate 112 material may become the path of least resistance for such eddy currents. Regardless whether eddy currents travel the longer path through the case portion or through the plate portion, the effect is to increase the resistance met by such eddy currents 41 in the case 30, thus reducing the attenuation of wireless communications.

Despite the foregoing examples, any overlap between the hole 116/plate 112 and the communication coil 40 will assist in reducing attenuation, even if such overlap is only partial, and even if the hole 116/plate 112 and coil 40 are not centered around the same axis 101, as shown in the different examples depicted in FIG. 5C. In other examples, the hole 116/plate 112 might comprise the entirety of the flat side 31b of the bottom case portion 30b, or the plate 112 might comprise the entirety of the bottom case portion 30b, although this isn't depicted.

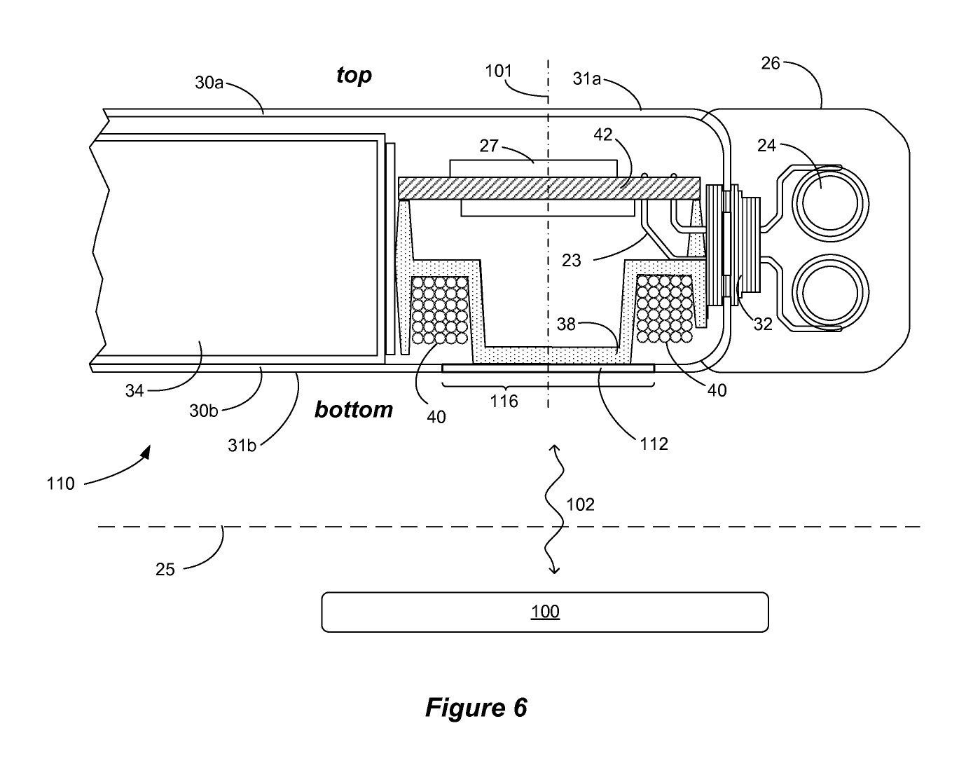

FIG. 6 shows the improved IPG 110 with the plate 112 in cross section. Note that the IPG 110 is implanted in the patient's tissue 25 with the bottom side 31b of the case out, which is preferred as communication coil 40 is placed closer to the bottom side 31b than to the top side 31a, and is thus in closer proximity to the external controller 100. This orientation of the IPG 110 in the patient also tends to prevent the IPG's PCB 42, which is proximate to the top case portion 30a, from interfering with communications 102 between the IPG 110 and the external controller 100.

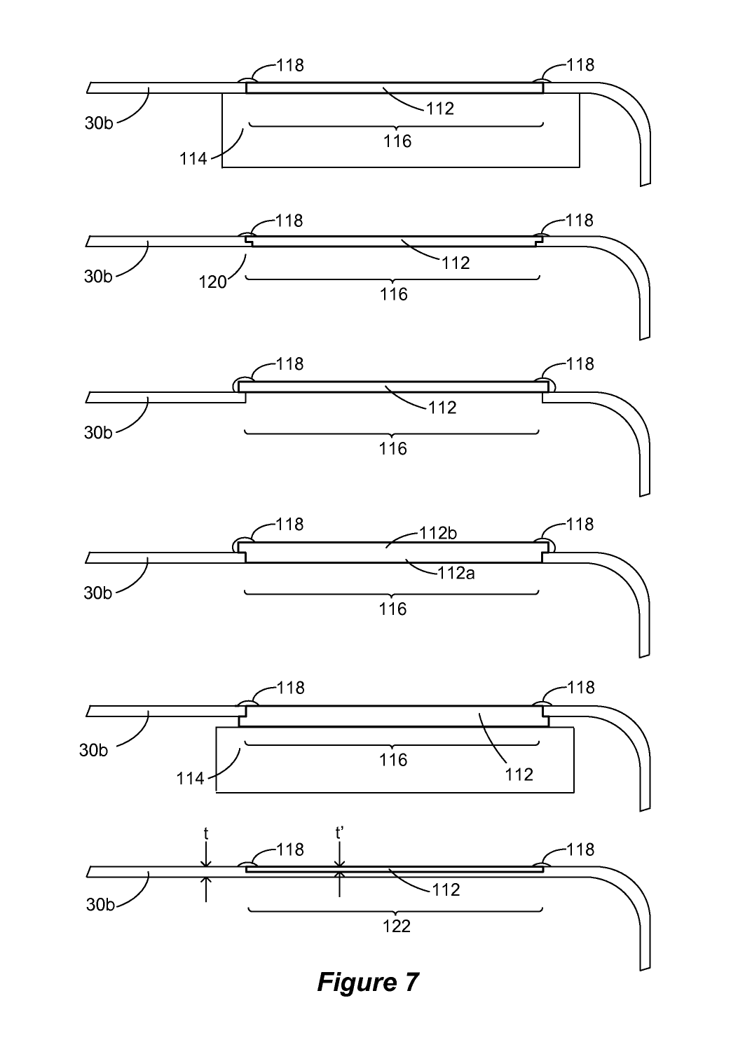

FIG. 7 shows different manners in which the plate 112 can be affixed to the bottom case portion 30b to cover hole 116. In the first example, the plate 112 is sized to match the hole 116 in the bottom case portion 30b, such that the outer surfaces of the bottom case portion 30b and the plate 112 are substantially flush (e.g., less than 10 mils difference). To prevent the plate 112 from falling through the hole 116 during its attachment, it is supported by a chuck 114, which may be made of high-temperature plastic for example and thus not affected by the laser welding to follow. Once the plate 112 is held in place within the hole 116 by the chuck 114, the seam between the plate 112 and the case portion 30b can be laser welded to form one or more welds 118, thus allowing the plate 112 to cover the hole 116 with a good hermetic seal. Laser welding is easily accomplished, especially considering that the materials for the plate 112 and the case portion 30b, while different, are similar enough to facilitate adhesion thought melting.

In the second example, the edges of the plate 112 and bottom case portion have been formed with interlocking steps 120. This holds the plate 112 in place during welding 118 without the need for chuck 114.

In the third example, plate 112 is slightly larger than hole 116, and thus sits on top of the hole during laser welding 118, again without falling through and without the need for chuck 114.

In the fourth example, plate 112 comprises a lower portion 112a design to fit the size of the hole 116, and an upper portion 112b which is slightly bigger than the hole 112b, which prevents it from falling though.

In the fifth example, the same plate 112 is used as in the fourth example, but is inserted from the inside of the bottom case portion 30. As a result, the plate 112 is substantially flush with the outside portion of the bottom case portion, which can then be laser welded 118. Chuck 114 is again helpful in holding the plate 112 in place during welding.

The last example shows a portion 122 of the bottom case portion 30b that has been thinned (t′) compared to the bulk thickness (t) of the case portion. As there is no hole 116 in the bottom case portion, the plate 112 will not fall through the bottom case portion 30b, and again no chuck 114 is needed. In this example, although some amount of higher-conductivity case portion material is present in portion 122, thinning of this material will still reduce attenuation of communications to some degree.

These are merely examples of how the plate 112 can be affixed to the bottom case portion 30b, and other manners are possible. For example, the plates 112 can be affixed from the inside of the case portion 30b (as in the fifth example), even though affixing from the outside has largely been depicted. As some of the examples show, the plate 112 can have a thickness equal to the thickness of the bottom case portion (t), although again this is not strictly necessary as some of the examples make clear. It should be noted that attenuation of communications will be benefitted by having the thinnest plate 112 possible (that still provides suitable strength and hermeticity) as a thinner plate 112 will lower plate conductivity even further. In this regard, it may be beneficial in other examples that the plate 112 be thinner than the bottom case portion (t). Further, while the area of the hole 116 and plate 112 are substantially equal in the depicted embodiments (varying by no more than 1% for example), the plate 112 can be substantially larger than the hole 116 in other examples.

Furthermore, while laser welding is the preferred manner for affixing the plate to the case portion, this also is not strictly necessary. Instead, the plate 112 may be affixed by brazing, or using a biocompatible and hermitic glue or other adhesive.

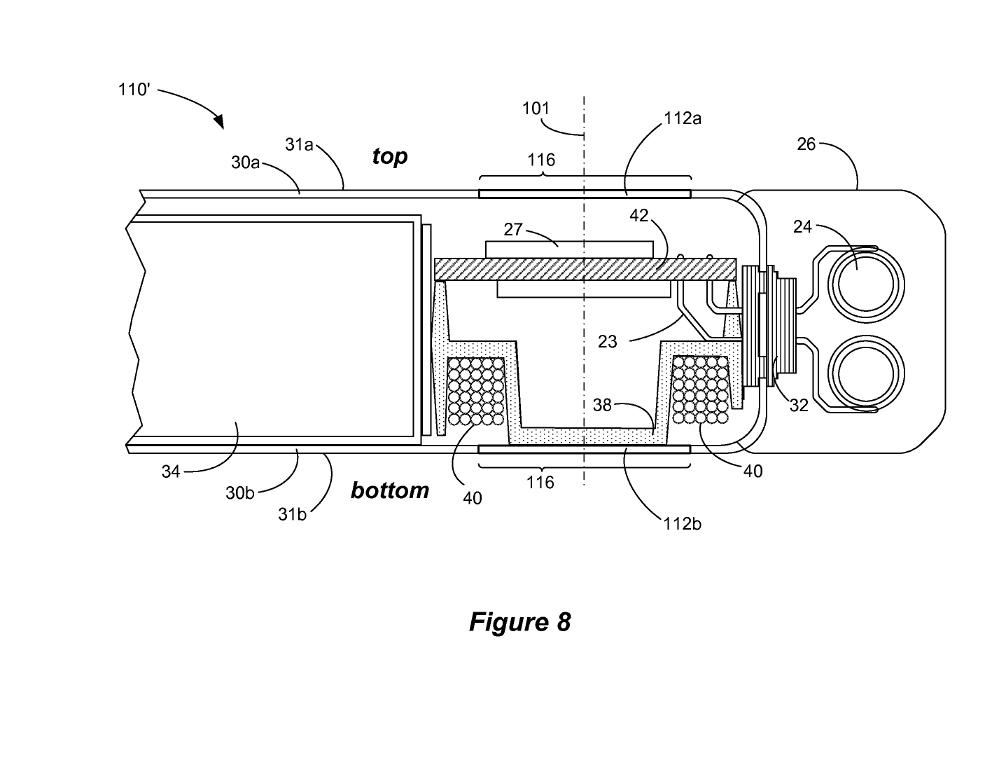

FIG. 8 shows another example of an improved IPG 110′ having two plates 112a and 112b of the type described earlier, one in the bottom case portion 30b and one in the top case portion 30a. As shown, the two plates 112a and 112b are centered along axis 101 and communication coil 40 as noted before. In this example, the additional plate assists in promoting flux through both sides 31a and 31b of the IPG 110′. This can be beneficial given the generally circular nature of flux lines with respect to the communication coil 40. Providing plates 112a and 112b on each side of the IPG 110′ can also allow either side of IPG 110′ to be oriented outward of the patient.

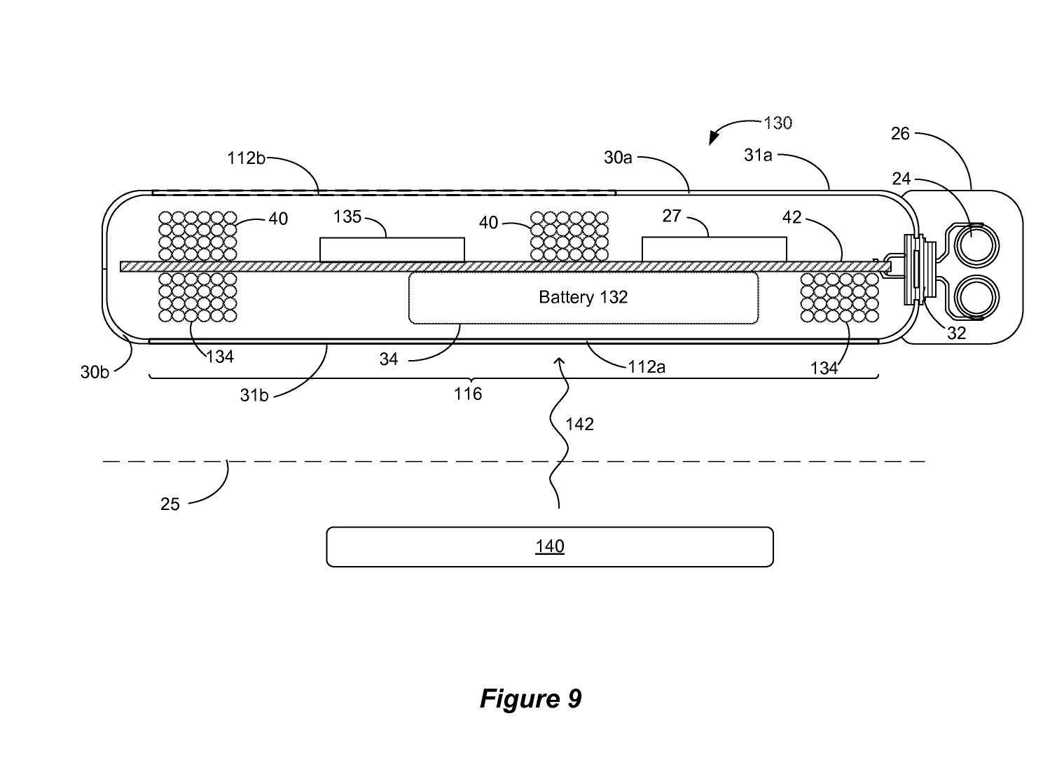

Although the addition of a metallic plate 112 to the case 30 of an IPG 110 has been developed with the primary goal of improving communications, plate 112 can also assist in wireless charging of the battery in the IPG. This is shown in FIG. 9, in which an external charger 140 is used to produce an AC magnetic field 142 to charge a battery 132 in IPG 130. This field 142 is received by a charging coil 134, and the AC current induced in this coil 134 is coupled to rectifier circuitry 135 that rectifies the current to a DC level that is used to charge the battery 132. See, e.g., U.S. Patent Application Publication 2013/0096651, discussing external chargers and battery charging in more detail. The AC magnetic charging field 142 is subject to the same concerns, namely attenuation in the case 30. As such, a plate 112a is positioned proximate to the charging coil 134. Because the charging coil 134 is wound around the entirety of the inside of the case, the plate 112a effectively comprises the entire flat surface of the bottom case portion 30b in this example.

IPG 130 additionally contains a separate communication coil 40 such as that described earlier. Even though it is not proximate to the plate 112a (it is proximate the top case portion 30a rather than the bottom case portion 30b to which the plate 112a is affixed), the communication coil 40 will still benefit from reduced attenuation in plate 112a. A secondary plate 112b used primarily for the benefit of the communication coil 40 could also be used, as shown in dotted lines. If communication coil 40 was proximate to the same side of the IPG as the charging coil 134, that side 31 or case portion might contain two holes 116 covered by two plates 112, although this is not depicted. Alternatively, although not depicted, either of the coils 40 or 134 could comprise a combined communication/charging coil coupleable to both rectifier circuitry 135 and modulation/demodulation 27 circuitry and thus capable of performing both functions.

It should be noted that plates 112 have been described as being particularly helpful when near-field magnetic induction is used as the means for wireless communications of data or a charging field, plates 112 are not limited in their utility to such means of communication. Indeed, plates 112, by virtue of their lower conductivities, will assist in reducing attenuation of far-field Radio Frequency (RF) means of communication, such as Bluetooth, Zigbee, Wifi, etc.

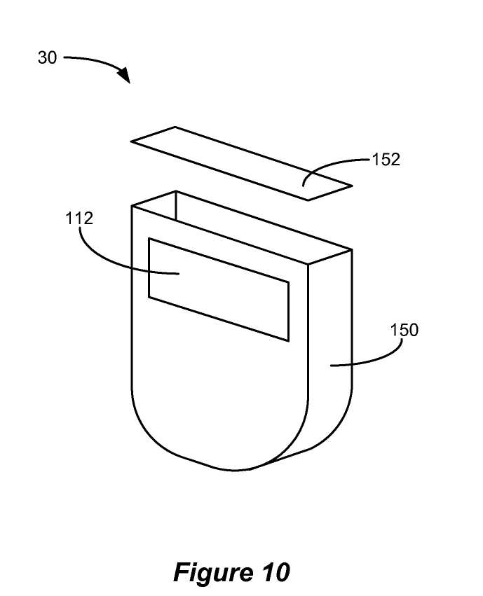

Although the case 30 is described as comprising two case portions 30a and 30b, one skilled will understand that the case 30, and use of the disclosed plate(s) 112, are not so limited. For example, and referring to FIG. 10, case 30 can also comprise a uniform structure generally resembling a “cup” 150 into which the internal components are positioned during assembly. A cap 152 can then be used to seal (e.g., weld) the open end of the cup. Although not shown, the cap 152 would likely have a feedthrough 32 surrounded by a header 26 as illustrated earlier. The disclosed plate(s) 112 can be used with such a case, or any other case construction, regardless of the number of pieces such case may comprise during assembly.

The following claims at times recite “a” structure, but this should not be construed as limiting scope to devices that only contain a singular one of such structures. For example, while the claims recite a case having “a” hole, and “a” plate, a case having two holes and corresponding plates would still be within the scope of the claims by virtue of any hole and its corresponding plates.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An implantable medical device, comprising:
a case formed of a first metallic material, the case having a first hole;
a first coil within the case, wherein the first coil is configured to send or receive a wireless signal to or from an external device, and wherein the first coil is centered with respect to the first hole; and
a first plate formed of a second metallic material, wherein the first plate is affixed to the case to cover the first hole, and wherein the conductivity of the second metallic material is lower than the conductivity of the first metallic material.

2. The device of claim 1, wherein the first plate is flat.

3. The device of claim 1, wherein the case comprises two case portions, and the first plate is affixed to one of the case portions.

4. The device of claim 1, wherein the case comprises first and second sides, and the first plate is affixed to the first side.

5. The device of claim 1, wherein the first plate is affixed to the case by one or more welds.

6. The device of claim 1, wherein the first plate is affixed to the case by one or more brazes.

7. The device of claim 1, wherein an outer surface of the first plate is substantially flush with an outer surface of the case.

8. The device of claim 1, wherein the case comprises a flat portion, and the first hole is located on the flat portion.

9. The device of claim 1, wherein the first plate comprises a plate formed solely of the second metallic material.

10. The device of claim 1, wherein the case comprises substantially parallel first and second sides, the first plate is affixed to the first side, and the first coil is closer to the first side than the second side.

11. The device of claim 1, wherein the first coil has an outer area, the first hole has an area, and the area of the first hole is equal to or greater than the outer area of the first coil.

12. The device of claim 1, wherein the first coil is coupled to one or more of modulation and demodulation circuitry within the case.

13. The device of claim 1, further comprising a battery within the case, wherein the first coil is coupled to rectifier circuitry within the case for providing power to the battery.

14. The device of claim 1, further comprising a header affixed to the case.

15. The device of claim 14, further comprising at least one lead connector within the header configured to couple to at least one electrode lead.

16. The device of claim 1, wherein the first conductive material comprises a titanium alloy, and wherein the second conductive material consists essentially of titanium.

17. The device of claim 1, wherein the case further has a second hole, and further comprising
a second coil within the case, wherein the second coil is configured to send or receive a wireless signal to or from an external device, and wherein the second coil is centered with respect to the second hole; and
a second plate formed of the second metallic material, wherein the second plate is affixed to the case to cover the second hole.

18. The device of claim 17, wherein the first coil comprises a telemetry coil and the second coil comprises a charging coil.

19. The device of claim 17, wherein the case comprises first and second portions, and wherein the first plate is affixed to the first case portion and the second plate is affixed to the second case portion.

20. The device of claim 17, wherein the case comprises first and second sides, and wherein the first plate is affixed to the first side and the second plate is affixed to the second side.

21. The device of claim 17, wherein the first and second plates are affixed to one side of the case.
